(19) United States
(12) Patent Application Publication
(10) Pub. No.: US 2005/0186115 A1
(43) Pub. Date: Aug. 25, 2005

(54) Scientific phenomenon evaluation device, pH measurement experimental device and manufacturing method of the device

(75) Inventors: Yasunori Ichikawa, Minami-Ashigara-shi (JP); Tomohide Ueyama, Minami-Ashigara-shi (JP); Fumiko Shiraishi, Minami-Ashigara-shi (JP); Tetsuo Kurahashi, Minami-Ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/059,391

(22) Filed: Feb. 17, 2005

(30) Foreign Application Priority Data
Feb. 20, 2004 (JP) NO.2004-45316

(51) Int. Cl. G01N 33/00

(52) U.S. Cl. 422/68.1; 73/64.56; 422/100

(57) ABSTRACT

According to the scientific phenomenon evaluation device of the present invention, a test liquid is injected into the first reservoir while sample liquids are injected into the second reservoirs. When the test liquid is supplied to the second reservoirs by being caused to flow through the branching-structure channel, the test liquid and the sample liquid mix and react with each other to cause, for example, a scientific phenomenon such that the colors of the sample liquid change. Thus, the plurality of sample liquids can be evaluated by injecting the test liquid one time. In this case, the scientific phenomenon can be grasped with a single glance since at least the scientific phenomenon in the second reservoirs is visually recognizable. Moreover, the scientific phenomenon evaluation device of the present invention can be effectively used as a portable pH measurement experimental device.

### BACKGROUND OF THE INVENTION

1. Field of the Invention

The present invention relates to a scientific phenomenon evaluation device, a pH measurement experimental device and a manufacturing method of the evaluation or experimental device. Especially, the present invention relates to a scientific phenomenon evaluation device and a pH measurement experimental device which are low-priced, which cause only a low environmental load, and which are suitable for easily enjoying high technology, and a method of manufacturing the evaluation or experimental device.

2. Description of the Related Art

Scientific phenomenon evaluation devices of various constructions including experimental devices for scientific education have been proposed (see Japanese Patent Application Laid-open No. 2000-242162).

For example, Japanese Patent Application Laid-open No. 2000-242162 discloses a scientific device for educational use which enables observation of natural phenomena relating to changes of water with respect to temperature by cooling or freezing water in a container or water vapor in the air, and which is simple in structure and capable of faithfully reproducing various natural phenomena relating to changes of water with respect to temperature.

As chemical experimental devices for educational uses, experimental kits such as “Kagaku to Gakushu Jikken Kit Series” (science and learning experimental kit series) and “Otonano Kagaku Chikyu Kankyo Bunseki Kit” (science for adults global environment analysis kit) from Gakken Co., Ltd. have been put on the market. Such experimental kits are being sold at a comparatively low price, about several hundred yen to three thousand yen, enabling children to dream about sciences and users to enjoy experiments and gaining public favor.

### SUMMARY OF THE INVENTION

However, conventional scientific phenomenon evaluation devices of this kind including the one described in Japanese Patent Application Laid-open No. 2000-242162 are comparatively complicated in construction, difficult to provide at a low price and inappropriate as an apparatus purchased by all pupils in a class.

Experimental kits comparatively simple in construction are usually low-priced and suitably as a kit purchased and used by all pupils in a class. However, those kinds of kits are usually unsatisfactory in terms of finish accuracy and therefore require increased amounts of chemicals. If such an experimental kit is used by all pupils in a class, an environmental load due to disposal of a waste liquid for example, which is not negligible, occurs.

The most of the conventional experimental kits are for experience of classical scientific experiments and only a few of them enable easily enjoying high technology.

Attention has recently been focused on environmental problems. Damage by acid rain, etc., have become a big concern. There is a demand for portable pH measurement experimental device easily carried and used to make a pH measurement on rain drops, a PH measurement on earth, a PH measurement on city water, etc.

In view of the above-described circumstances, an object of the present invention is to provide a scientific phenomenon evaluation device and a pH measurement experimental device which are low-priced, which cause only a low environmental load, and which are suitable for easily enjoying high technology, and a manufacturing method of the evaluation or experimental device.

To achieve the above-described object, a first aspect of the present invention provides a scientific phenomenon evaluation device, comprising: a first reservoir into which a test liquid is injected, a plurality of second reservoirs into which sample liquids are injected, a fine branching-structure channel having a sectional area of 1 mm² or less, the branching-structure channel providing communication between the first reservoir and the second reservoirs, and a flow rate distribution device which uniformly distributes the rates of flow of the test liquid supplied from the first reservoir to the second reservoirs, wherein at least a scientific phenomenon in the second reservoirs is visually recognizable.

According to the first aspect of the present invention, a test liquid is injected into the first reservoir while sample liquids are injected into the second reservoirs. When the test liquid is supplied to the second reservoirs by being caused to flow through the branching-structure channel, the test liquid and the sample liquid mix and react with each other to cause, for example, a scientific phenomenon such that the colors of the sample liquid change. Thus, the plurality of sample liquids can be evaluated by injecting the test liquid one time. In this case, the scientific phenomenon can be grasped with a single glance since at least the scientific phenomenon in the second reservoirs is visually recognizable. It is more preferable to make the first reservoir and the entire branching-structure channel visually recognizable as well as the second reservoirs.

The portions referred to as “Reservoir” are provided as voids normally empty. When the evaluation device is operated, chemicals or the like are supplied to the reservoirs.

In the first aspect of the present invention, the branching-structure channel for supplying a test liquid injected into the first reservoir to sample liquids injected into the second reservoirs is required. In supply of the test liquid to the second reservoirs, the amount of the test liquid supplied to one of the second reservoirs with a longer channel length from the branching point is reduced relative to the amount of test liquid supplied to another of the second reservoirs with a shorter channel length. In the present invention, however, the flow rate distribution device for uniformly distributing the rates of flow of the test liquid flowing from the first reservoir to the second reservoirs is provided to enable equal amounts of the test liquid to the second reservoirs.

The branching-structure channel of the evaluation device is formed as a fine channel having a sectional area of 1 mm² or less. Therefore, accuracy high enough to experience high technology can be obtained and the amounts of test and sample liquids used are small, so that the environmental load is small.

The sectional area of the branching-structure channel is 1 mm² or less, more preferably 0.0025 to 0.64 mm², and most preferably 0.01 to 0.25 mm². The capacity of the first reservoir and the second reservoirs is preferably in the range from 5 to 5000 mm³.

Preferably, the flow rate distribution device comprises a restriction member provided in each of branching channel portions downstream of the branching point in the branching-structure channel, and the amounts of restrictions by the restriction members are varied among the branching channel portions. The rates of flow in the branching channel portions can be changed by selecting the amounts of restrictions by means of the restriction members provided in the branching channel portions of the branching-structure channel. In this way, equal amounts of the test liquid can be supplied to the sample liquids in the second reservoirs even if the channel portions between the branching point and the second reservoirs differ in channel length. As the restriction member, an orifice can be suitably used.

Preferably, in another example of the flow rate distribution device, the flow rate distribution device is formed in a channel structure in which portions between the first reservoir and intermediate points in the branching-structure channel are formed of capillary channels, and in which the portions between the downstream ends of the capillary channels and the second reservoirs are formed so as to be uniform in channel length. If the portions between the first reservoir and intermediate points in the branching-structure channel are formed of capillary channels, the test liquid injected into the first reservoir fills the capillary channels by capillary action, and equal amounts of the test liquid can be supplied to the sample liquids in the second reservoirs since the channel portions between the downstream ends of the capillary channels and the second reservoirs are uniform in channel length.

Preferably, the evaluation device of the first aspect includes a base plate in which one groove, a plurality of grooves branching off from the one groove, the first reservoir and the second reservoirs are formed, and a cover plate placed close to the surface of the base plate to cover the grooves and to thereby form the branching-structure channel in the base plate, and at least one of the base plate and the cover plate is transparent. If the evaluation device is constituted by such base and cover plates, manufacturing of the device is facilitated. If at least one of the base plate and the cover plate is transparent, a scientific phenomenon in the branching-structure channel can be visually recognized.

Preferably, through-holes capable of communication between external air and the first reservoir and the second reservoirs are formed in the cover plate. If such holes capable of communication between external air and the first reservoir and the second reservoirs are formed to provide communication between the reservoirs and external air, the holes can be used as inlets for introducing liquids into the branching-structure channel and as air vent holes. The facility with which phenomena which occur in the microchannels are controlled is also improved thereby.

To achieve the above described object, a second aspect of the present invention provides a pH measurement experimental device, wherein the device is a portable experimental device for pH measurement by the scientific phenomenon evaluation device of the first aspect.

The scientific phenomenon evaluation device having the structure of the first aspect can be effectively used as a portable pH measurement experimental device. However, the evaluation device of the present invention is not limited to use for pH measurement only. The evaluation device of the present invention can be applied to evaluation of various chemical phenomena liquids caused by mixing of a test liquid with sample liquids, e.g., acid-alkali reaction and hydrolysis reaction.

To achieve the above-described object, a third aspect of the present invention provides a manufacturing method of the scientific phenomenon evaluation device of the first aspect, comprising the steps of: applying a resin material to a surface of a reverse mold having a surface in which a shape reverse to the shape of the grooves in the base plate is formed, setting the resin material, and releasing the set resin material from the reverse mold to form the base plate.

To achieve the above-described object, a fourth aspect of the present invention provides a manufacturing method of the pH measurement experimental device of the second aspect, comprising the steps of: applying a resin material to a surface of a reverse mold having a surface in which a shape reverse to the shape of the grooves in the base plate is formed, setting the resin material, and releasing the set resin material from the reverse mold to form the base plate.

According to the third and the fourth aspect of the present invention, the base plate is formed by transfer molding using a reverse mold having a surface in which a shape reverse to the shape of the grooves in the base plate is formed, and can therefore be provided with accuracy a low cost, thereby enabling the evaluation device to be manufactured at a low cost. While this method includes “applying a resin material to a surface of a reverse mold and setting the resin material”, a method including bringing a resin material into contact with a surface of a reverse mold and transferring the shape of the grooves into the resin material surface with a hot press or the like and other methods are based on the same technical ideal and can be said to be within the equivalent scope of the present invention.

As described above, the present invention enables easily enjoying high technology at a low cost while reducing the environmental load.

### BRIEF DESCRIPTION OF THE DRAWINGS

FIGS. 1A and 1B are a plan view and a schematic sectional view, respectively, of the construction of a pH measurement experimental device in accordance with the present invention in a case where a restriction member is provided as a flow rate distribution device;

FIGS. 2A and 2B are a plan view and a schematic sectional view, respectively, of an apparatus obtained by replacing the flow rate distribution device shown in FIGS. 1A and 1B with a capillary channel system;

FIGS. 3A to 3D are schematic sectional views showing a pH measurement procedure; and

FIGS. 4A to 4D are diagrams for explaining a case where a test liquid is fed from the first reservoir to the second reservoirs by using expansion of a gas.

### DETAILED DESCRIPTION OF THE PREFERRED EMBODIMENTS

A preferred embodiment of a scientific phenomenon evaluation device, a pH measurement experimental device and a manufacturing method of the evaluation or experimental device will be described in detail with reference to the accompanying drawings.

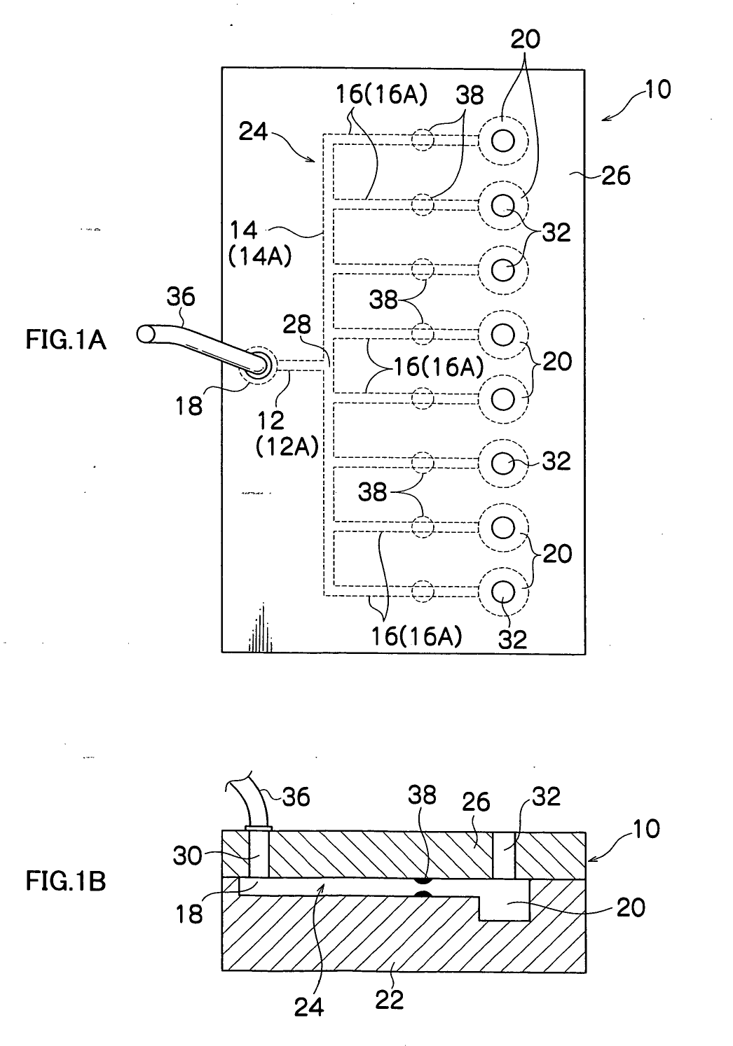

FIG. 1A is a plan view of the construction of a pH measurement experimental device 10 as a preferred from of a scientific phenomenon evaluation device in accordance with the present invention. FIG. 1B is a schematic sectional view of FIG. 1A.

The pH measurement experimental device 10 is constituted mainly of a base plate 22 in which grooves 12, 14, and 16 are formed as one groove and a plurality of grooves branching off from the one groove, and in which a first reservoir 18 and a plurality of second reservoirs 20 are also formed, and a cover plate 26 placed close to the surface of the base plate 22 to cover the grooves 12, 14, and 16 and to thereby form a branching-structure channel 24 in the base plate 22.

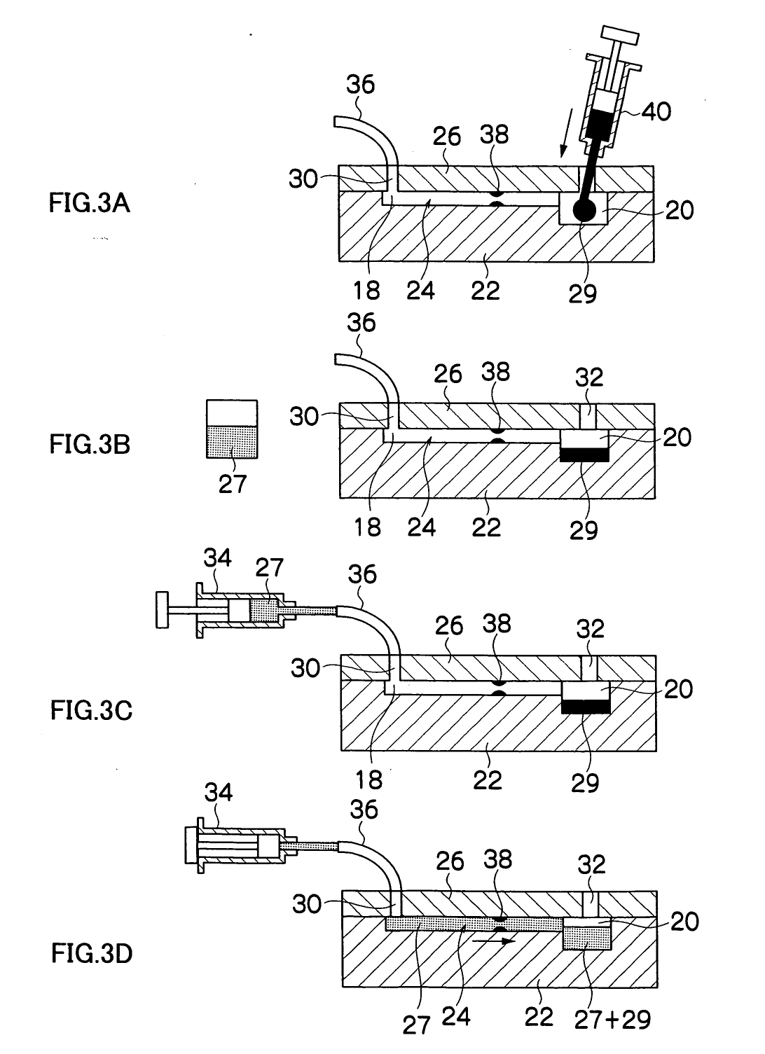

That is, a first channel 12A extends from the first reservoir 18 into which a test liquid 27 is injected (see FIG. 3); second channels 14A perpendicular to the first channel 12A branch off at a branching point 28 in opposite directions; and a plurality of third channels 16A respectively extend from the branching second channels 14A to the second reservoirs 20 in parallel with each other, thus forming the branching-structure channel 24. The branching-structure channel 24 is formed as a fine channel having a sectional area of 1 mm² or less. Further, a first through-hole 30 for communication to external air is formed in a portion of the cover plate 26 facing the first reservoir 18, and a plurality of second through-holes 32 for communication to external air are also formed in portions of the cover plate 26 facing the second reservoirs. A tube 36 to which a device for injecting the test liquid 27 into the first through-hole 30, e.g., a dropping syringe or an injector 34 (see FIG. 3) is coupled is connected to the first through-hole 30.

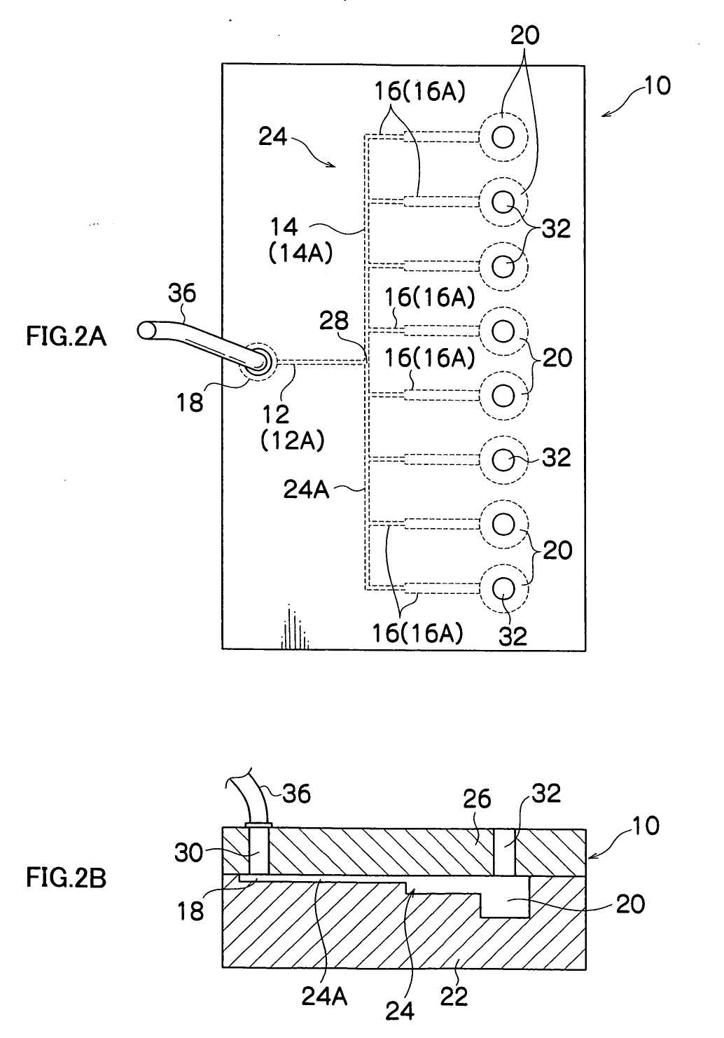

In the third channels 16A forming the branching-structure channel 24, flow rate distribution devices for uniformly distributing flow rates at which the test liquid 27 flows from the first reservoir 18 to the second reservoirs 20 are respectively provided. As each flow rate distribution device, a type of device using a restriction member or a type of device using capillary action can be suitably used. FIGS. 1A and 1B shows a case where a type of device using a restriction member is incorporated. FIGS. 2A and 2B shows a case where a type of device using capillary action is incorporated.

The flow rate distribution device using the restriction member 38 will be described with reference to FIGS. 1A and 1B. The restriction members 38 provided in the third channels 16A are set so that the amount of restriction by the restriction member 38 is reduced with increasing channel length between the branching point 28 and the second reservoirs 20. The test liquid 27 is thereby enabled to flow more easily in one of the channels than in another of the channels shorter in length between the branching point 28 and the second reservoirs 20. It is possible to uniformly distribute the rates of flow of the test liquid 27 from the first reservoir 18 to the second reservoirs 20 by adjusting the amounts of restriction by the restriction members 38 to suitable values.

The flow rate distribution device using capillary action will be described with reference to FIGS. 2A and 2B. A capillary channel 24A formed from the first reservoir 18 to intermediate points in the third channels 16A, and the channel portions between the downstream ends of the capillary channel 24A and the second reservoirs 20 are formed so as to be uniform in channel length. That is, forming the capillary channel 24A so that the channel portions between the downstream ends of the capillary channel 24A and the second reservoirs 20 are uniform in channel length may suffice. The test liquid 27 injected into the first reservoir 18 fills the thus-formed capillary channel 24A by capillary action, so that a common starting line from which the test liquid 27 is supplied to the second reservoirs 20 is set. If in this state a liquid feed force is applied to the test liquid 27, the rates of low of the test liquid from the first reservoir 18 to the second reservoirs 20 can be uniformly distributed.

There are no particular restrictions on the selection of the planar size of the base plate 22 and the cover plate 26 for manufacture of the pH measurement experimental device 10. However, the base plate 22 and the cover plate 26 may provided in a potable size, e.g., 80×50 mm according to the characteristics of the pH measurement experimental device used in a school. Also, there are no particular restrictions on the selection of the thickness of the base plate 22 and the cover plate 26. However, the thickness of each of the base plate 22 and the cover plate 26 may be set to, for example, about 5 mm by considering the strength, economy and other factors.

There are no particular restrictions on the selection of the material of the base plate 22. However, a resin material, more specifically polydimethylsulfoxide (PDMS), polymethyl methacrylate (PMMA), polyvinyl chloride (PVC), an ultraviolet curing resin, a thermosetting resin, polycarbonate (PC) or the like is preferably used.

In the case where the restriction member 38 shown in FIGS. 1A and 1B is used, the sectional area of the grooves 12, 14, and 16 formed in the surface of the base plate 22 is 1 mm² or less, as mentioned above. The sectional area of the grooves 12, 14, and 16 is more preferably 0.0025 to 0.64 mm², and most preferably 0.01 to 0.25 mm².

In the case where capillary action is used as shown in FIGS. 2A and 2B, the sectional area of the grooves 12, 14, and 16 is 1 mm² or less. In this case, since there is a need to generate capillary action in the capillary channel 24A portions, the sectional area of the capillary channel 24A is 90% or less, preferably 75% or less, and particularly preferably 50% or less of the main channels (the channels other than the capillary channel 24A). The channel portions other than the capillary channel 24A are the same as those in the case shown in FIG. 1.

The capacity of the first reservoir 18 and the second reservoirs 20 is preferably in the range from 5 to 5000 mm³.

There are no particular restrictions on the selection of the sectional shape of the grooves 12, 14, and 16. Any of various shapes such as a rectangular shape (a square, a rectangle), a trapezoidal shape, a V-shape and a semicircular shape can be adopted. However, a rectangular shape (a square, a rectangle) is preferred from the viewpoint of facilitating manufacturing based on a manufacturing method described below.

There are no particular restrictions on the selection of the material of the cover plate 26. However, it is preferable to use a transparent plate as the cover plate 26 to enable a scientific phenomenon in the channel 24 to be visually recognized. Such a plate usable as the cover plate 26 is, for example, a plate formed of any of various resin materials, e.g., polydimethylsulfoxide (PDMS), polymethyl methacrylate (PMMA), polyvinyl chloride (PVC), an ultraviolet curing resin and polycarbonate (PC), a film of any of various resins, e.g., polyethylene terephthalate (PET), polyethylene naphthalate (PEN) and triacetyl cellulose (TAC), or any of various glasses (soda-lime glass, borosilicate glass, etc.).

Ordinarily, the cover plate 26 is a flat plate having flat front and back surfaces. However, the cover plate 26 may be formed in such a manner that the front surface corresponding to the fine branching-structure channel 24 is formed as a convex lens having a cylindrical surface to enable a scientific phenomenon to be observed in an enlarged state.

An arrangement may alternatively be adopted in which the base plate 22 is transparent while the cover plate 26 is nontransparent.

It is preferable to ensure a sufficiently high degree of flatness of the front surface of the base plate 22 (the surface in which the grooves are formed) and the back surface of the cover plate 26 (the surface maintained in close contact with the base plate 22) from the viewpoint of the formation of the branching-structure channel 24 and prevention of liquid leakage.

A method of forming the base plate 22 will now be described. A reverse mold having a surface in which a shape reverse to that of the grooves 12, 14, and 16 is formed is first prepared. It is necessary to also form shapes reverse to those of the first reservoir 18 and the second reservoirs 20 in the surface of the reverse mold. As a method of manufacturing this reverse mold, any of well-known working methods such as machining with a machining center or the like, electro-discharge machining, ultrasonic machining and photoetching can be used.

A mold release agent is applied to the surface of the reverse mold. This mold release agent may be selected according to the kind of the resin material forming the base plate 22, machining conditions (including temperature) and other factors.

A resin material is thereafter applied to the surface of the reverse mold and is set. If the resin material is, for example, an ultraviolet curing resin, it is set by being irradiated with ultraviolet rays. If the resin material is, for example, a thermoplastic resin such as polyvinyl chloride, the resin material is brought into contact with the surface of the reverse mold and is formed by thermal transfer molding using a hot press machine.

The set resin material is released from the reverse mold.

By this method, the grooves 12, 14, and 16 can be formed with accuracy at a low cost and the evaluation device can be manufactured at a low cost.

A method of using the pH measurement experimental device in accordance with the present invention will be described. It is necessary to provide a set of members (1) to (12) described below as the pH measurement experimental device 10.
1) The reverse mold
2) The resin material for the base plate 22
3) A mold frame for molding the base plate 22 (a mold frame used when the resin is caused to flow at the time of molding of the base plate 22)
4) The cover plate 26
5) An injector for injecting a liquid (An injector used to inject necessary liquids into the first reservoir 18 and the second reservoirs 20 according to a test purpose. One injector may be used for each of chemicals to be injected, or one injector may be used for injection of a plurality of chemicals by being cleansed.) In the case of a device for school education use, any device other than injectors capable of supplying a liquid to each reservoir may be used by considering the price, safety, etc., and an injection device such as a dropper may be used. However, a description will be made below with respect to an example of the device using an injector.
6) A tape for sealing the first through-hole

(A tape used as a cover over the first reservoir 18 after a liquid has been supplied to the first reservoir 18. It can also be used as a cover over the second reservoirs 20.)
7) A needle

(A needle used when required to form a hole in the sealing tape to allow air to flow into the first reservoir 18 and second reservoirs 20 corresponding to a change in the amount of a liquid when the liquid is fed forward or received in a case where a chemical or a sample is supplied or recovered.)
8) A casing

(A casing is attached for the purpose of preventing liquid leakage through the gap between the cover plate 26 and the base plate 22 or preventing damage to the cover plate 26 for example when this experimental set is assembled. This casing may comprise any of devices for various functions according to experimental purposes, e.g., a magnifying lens for facilitating observation of the channel.)
9) A liquid feed device

(A device by feeding a liquid by volumetric gas expansion in the first reservoir 18 may be used as well as a device such as an injector or a dropper for feeding a liquid by using the principle of a pump. This device for feeding a liquid by volumetric expansion operates in such a manner that when heat is applied to the first reservoir 18 in a state where the first reservoir 18 is covered with the sealing tape (when a finger tip is applied to the tape to perform heating by body heat for example), volumetric expansion of a liquid and/or a gas in the first reservoir 18 is caused. This is a method of feeding a liquid by using such a phenomenon.
10) A test liquid including a pH indicator

(As test liquid 27, a phenolphthalein solution or the like may be used.)
11) Experimental manual and pH limit samples

A manual in which descriptions of phenomena which can be learnt from this set, including a description of the purpose of a pH measurement made with this set, an explanation of phenomena and a description of applications, are made is attached according to need. The pH limit samples are color samples indicating the relationship between colors and pH values.
12) An experimental method procedure guide

This set enables pupils to make the base plate 22 by themselves. If making of the base plate 22 is omitted, the completed base plate 22 may be included instead of the members (1) to (3).

A pH measurement using this set of pH measurement experimental device 10 will be described in detail.

FIGS. 3A to 3D are schematic sectional views showing the procedure of an experimental method. A pH experimental device 10 using the restriction members shown in FIGS. 1A and 1B was used.

As shown in FIG. 3A, predetermined amounts of various sample liquids 29 on which pH measurements are to be made are injected into the second reservoirs 20 with an injector 40 for injecting sample liquids 29. A dropper, a pipette or the like may be used instead of the injector 40 to inject sample liquids 29. A pH measurement can be made even if sample liquids 29 are not injected to all the second reservoirs 20. Subsequently, a test liquid 27 including a pH indicator is prepared and drawn into an injector 34, as shown in FIG. 3B, and the injection tip of the injector 34 is inserted into the tube 36, as shown in FIG. 3C. The test liquid 27 is injected into the first reservoir 18 to be supplied to the second reservoirs 20 through the branching-structure channel 24, as shown in FIG. 3D. A dropper, a pipette or the like may be used instead of the injector 34 to inject the test liquid 27. The test liquid 27 is thereby caused to mix and react with the sample liquids in the second reservoirs 20 to exhibit colors according to the pH of the sample liquids 29. The colors of the sample liquids 29 are compared with the pH limit samples provided in the set to evaluate the pH of the sample liquids 29. In this case, since the restriction members 38 are provided in the plurality of third channels 16A to enable supply of equal amounts of the test liquid 27 to the sample liquids 29 as described above, the pH of each of the sample liquids 29 injected into the second reservoirs 20 can be correctly evaluated.

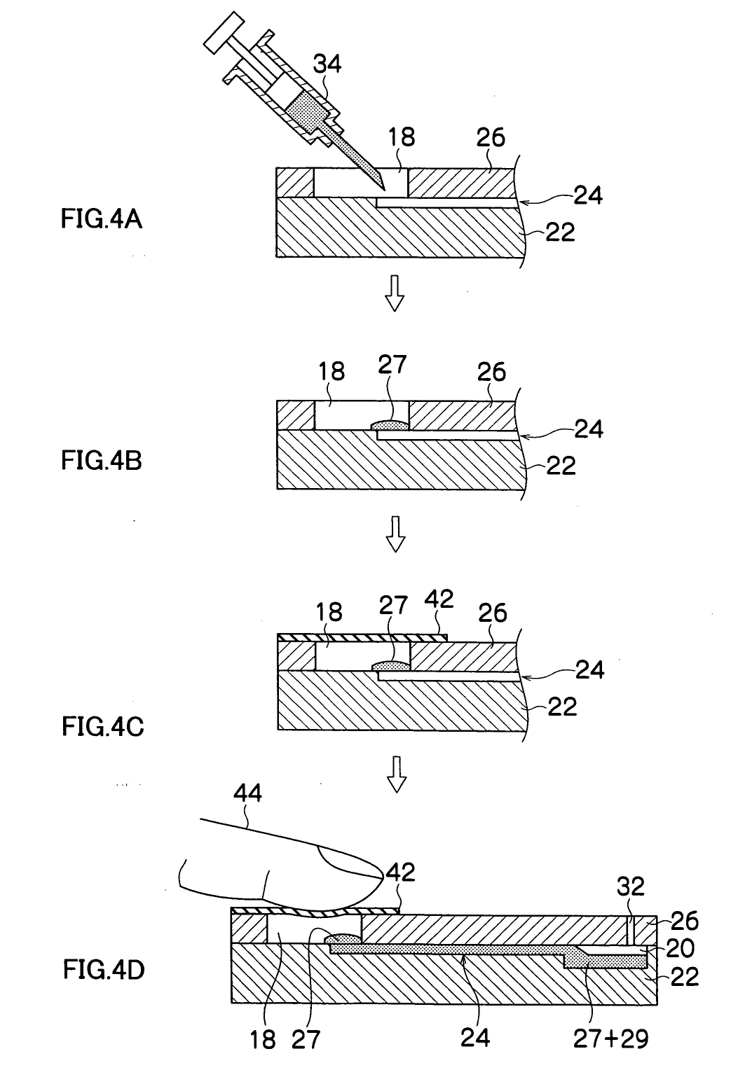

While the method of feeding of a liquid from the first reservoir 18 to the second reservoirs 20 using the pump principle of the injector 34, feeding of a liquid may be performed by using volumetric expansion of the liquid and/or a gas in the first reservoir 18, as shown in FIGS. 4A to 4D. FIGS. 4A to 4D show a case where the first reservoir 18 is provided in the cover plate 26 and the first through-hole 30 is not used.

When injection of sample liquids 29 into the plurality of second reservoirs 20 is completed, the test liquid 27 is injected into the first reservoir 18 with the injector 34 for example, as shown in FIGS. 4A and 4B. Subsequently as shown in FIG. 4C, the first reservoir 18 is covered by sealing the surface of the first reservoir 18 with the sealing tape 42. One side (a lower surface as viewed in the figure) of the sealing tape 42 is coated with a pressure-sensitive adhesive. The first reservoir 18 is thereby isolated from external air. Subsequently, a finger tip 44 is brought into contact with the upper surface of the sealing tape 42, as shown in FIG. 4D. A liquid feed device is thereby formed at the first reservoir 18. This liquid feed device operates in such a manner that volumetric expansion of a gas in the first reservoir 18 is caused by heat from the finger tip 44 to feed the test solution 27 to the second reservoirs 20 through the branching-structure channel 24.

In the arrangement shown in FIG. 4D, the liquid feed device may alternatively be such that the sealing tape 42 is pressed with the finger tip 44 to be depressed downward and the volume of the first reservoir 18 is thereby reduced to feed the test liquid into the branching-structure channel.

To facilitate observation of these phenomena, a magnifying glass or the like may be used. Also, the portion of the cover plate 26 corresponding to the branching-structure channel 24 may have a magnifying glass function (lens function), as described above.

The above-described pH measurement experimental device 10 has its essential portion formed as simply as possible to be provided at a low price while ensuring high experimental accuracy for the purpose of enabling children to perform scientific experiments in a micro field enjoying dreams about sciences. The device is capable of simultaneously measuring the pH values of a plurality of sample liquids 29 by injecting test liquid 27 one time. The device is also capable of a pH experiment with an extremely small amount of indicator or the like and limiting the amounts of waste liquids and materials after the completion of a test, thus reducing the environmental load. Further, the branching-structure channel 24 is formed as a fine channel on the order of microns to enable experience of a testing device using micro-nanotechnology.

The present invention has been described with respect to an example of the pH measurement experimental device 10, which is an example of the scientific phenomenon evaluation device. However, the present invention is not limited to the example. The device of the present invention can be used as a device for evaluating various phenomena such as chemical phenomena and physical phenomena of liquids caused by mixing of test liquid 27 with sample liquids 29, e.g., liquid diffusion phenomena, liquid thermal transfer phenomena, liquid mixing phenomena and liquid chemical phenomena (e.g., acid-alkali reaction and hydrolysis reaction).

While the invention has been described with respect to the case where the branching-structure channel 24, the first reservoir 18 and the second reservoirs 20 are formed in the base plate 22 and where the first through-hole 30 and the second through-holes 32 are formed in the cover plate 26, other arrangements are also possible. For example, an arrangement such as the one described above with reference to FIGS. 4A to 4D, in which the first reservoir 18 is formed in the cover plate 26 while the second reservoirs 20 are formed in the base plate 22, can be adopted.

In this embodiment, the injectors 34 and 40 are used to supply an indicator and samples to the reservoirs 18 and 20. However, as mentioned above, droppers, microsyringes or the like having the same functions as those of the injectors may be used instead of the injectors. It is ordinarily desirable to use a low-priced dropper in a scientific experimental device for educational use. In some cases, it is desired to use injectors according to a certain testing purpose, as described above.

1. A scientific phenomenon evaluation device, comprising:
a first reservoir into which a test liquid is injected;
a plurality of second reservoirs into which sample liquids are injected;
a fine branching-structure channel having a sectional area of 1 mm² or less, the branching-structure channel providing communication between the first reservoir and the second reservoirs; and
a flow rate distribution device which uniformly distributes the rates of flow of the test liquid supplied from the first reservoir to the second reservoirs,
wherein at least a scientific phenomenon in the second reservoirs is visually recognizable.

2. The scientific phenomenon evaluation device according to claim 1, wherein the flow rate distribution device comprises a restriction member provided in each of branching channel portions downstream of a branching point in the branching-structure channel, the amounts of restrictions by the restriction members being varied among the branching channel portions.

3. The scientific phenomenon evaluation device according to claim 1, wherein the flow rate distribution device is formed in a channel structure in which portions between the first reservoir and intermediate points in the branching-structure channel are formed of capillary channels, and in which the portions between the downstream ends of the capillary channels and the second reservoirs are formed so as to be uniform in channel length.

4. The scientific phenomenon evaluation device according to claim 1, further comprising:
a base plate in which one groove, a plurality of grooves branching off from the one groove, the first reservoir and the second reservoirs are formed; and
a cover plate placed close to the surface of the base plate to cover the grooves and to thereby form the branching-structure channel in the base plate,
wherein at least one of the base plate and the cover plate is transparent.

5. The scientific phenomenon evaluation device according to claim 2, further comprising:
a base plate in which one groove, a plurality of grooves branching off from the one groove, the first reservoir and the second reservoirs are formed; and
a cover plate placed close to the surface of the base plate to cover the grooves and to thereby form the branching-structure channel in the base plate,
wherein at least one of the base plate and the cover plate is transparent.

6. The scientific phenomenon evaluation device according to claim 3, further comprising:
a base plate in which one groove, a plurality of grooves branching off from the one groove, the first reservoir and the second reservoirs are formed; and
a cover plate placed close to the surface of the base plate to cover the grooves and to thereby form the branching-structure channel in the base plate,
wherein at least one of the base plate and the cover plate is transparent.

7. The scientific phenomenon evaluation device according to claim 4, wherein through-holes capable of communication between external air and the first reservoir and the second reservoirs are formed in the cover plate.

8. The scientific phenomenon evaluation device according to claim 5, wherein through-holes capable of communication between external air and the first reservoir and the second reservoirs are formed in the cover plate.

9. The scientific phenomenon evaluation device according to claim 6, wherein through-holes capable of communication between external air and the first reservoir and the second reservoirs are formed in the cover plate.

10. A pH measurement experimental device, wherein the device is a portable experimental device for pH measurement by the scientific phenomenon evaluation device of claim 1.

11. A pH measurement experimental device, wherein the device is a portable experimental device for pH measurement by the scientific phenomenon evaluation device of claim 2.

12. A pH measurement experimental device, wherein the device is a portable experimental device for pH measurement by the scientific phenomenon evaluation device of claim 3.

13. A pH measurement experimental device, wherein the device is a portable experimental device for pH measurement by the scientific phenomenon evaluation device of claim 8.

14. A pH measurement experimental device, wherein the device is a portable experimental device for pH measurement by the scientific phenomenon evaluation device of claim 9.

15. A manufacturing method of the scientific phenomenon evaluation device of claim 1, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

16. A manufacturing method of the scientific phenomenon evaluation device of claim 2, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

17. A manufacturing method of the scientific phenomenon evaluation device of claim 3, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

18. A manufacturing method of the scientific phenomenon evaluation device of claim 8, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

19. A manufacturing method of the scientific phenomenon evaluation device of claim 9, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

20. A manufacturing method of the pH measurement experimental device according to claim 10, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

21. A manufacturing method of the pH measurement experimental device according to claim 11, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

22. A manufacturing method of the pH measurement experimental device according to claim 12, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

23. A manufacturing method of the pH measurement experimental device according to claim 13, comprising the steps of:
applying a resin material to a surface of a reverse mold having a surface in which a shape reverse to the shape of the grooves in the base plate is formed;
setting the resin material; and
releasing the set resin material from the reverse mold to form the base plate.

24. A manufacturing method of the pH measurement experimental device according to claim 14, comprising the steps of:
applying a resin material to a surface of a reverse mold having a surface in which a shape reverse to the shape of the grooves in the base plate is formed;
setting the resin material; and
releasing the set resin material from the reverse mold to form the base plate.
